(19) United States
(12) Patent Application Publication
(10) Pub. No.: US 2007/0123748 A1
(43) Pub. Date: May 31, 2007

(54) Robot for minimally invasive interventions

(76) Inventor: Dwight Meglan, Westwood, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/487,153

(22) Filed: Jul. 14, 2006

Related U.S. Application Data
(60) Provisional application No. 60/699,087, filed on Jul. 14, 2005.

(51) Int. Cl. A61B 1/00; A61B 5/00; A61B 17/00

(52) U.S. Cl. 600/104; 606/1; 600/309

(57) ABSTRACT

The present invention relates to a miniature robotic device to be introduced, in the case of the heart, into the pericardium through a port, attach itself to the epicardial surface, and then, under the direct control of the user or physician, travel to the desired location for diagnosis or treatment.

### CROSS REFERENCE TO RELATED APPLICATIONS

This application claims the priority of U.S. Provisional Application No. 60/699,087 filed Jul. 14, 2005 entitled, ROBOT FOR MINIMALLY INVASIVE INTERVENTIONS. The entire content of the above application is being incorporated herein by reference.

### BACKGROUND OF THE INVENTION

Heart surgery is typically done by opening the chest cavity or by a minimally invasive procedure using the intercostal spacing to access the heart, or endoscopically in which surgical tools can be introduced via an endoscope channel.

Closed-chest endoscopic visualization of the epicardium utilizes techniques for evaluation of blunt chest trauma, pericardial effusion, lung cancer, staging, and epicardial implantation of ventricular pacing leads. Endoscope access can require thoracotomy with breach of the left pleural space. Direct access to the pericardial space via subxiphoid puncture is an increasingly practiced technique for epicardial procedures. In such procedures, catheter manipulation is guided solely by fluoroscopy.

The challenges of minimally invasive access are further complicated by the goal of avoiding cardiopulmonary bypass. Achieving this goal necessitates surgery on a beating heart. Thus instrumentation is needed that allows stable manipulation of tools at a location on the epicardium while the heart is beating. Local immobilization of the heart is the approach generally followed, utilizing endoscopic or open chest stabilizers that operate with mechanical pressure or suction. A continuing need exists for improvements in diagnostic and surgical devices which reduce invasiveness and improve beating heart surgery, thereby reducing risk and recovery time of the patient.

### SUMMARY OF THE INVENTION

The present invention relates to a miniature robotic device that is endoscopically introduced into an area of the body including, for example, the region of the abdominal cavity such as the pericardium or heart, body lumens such as the lungs or gastrointestinal tract, or regions of the spine or brain. The robotic device is attached to the epicardial or other surface. A user than controls the movement and operation of the device to perform diagnostic and/or therapeutic functions. The robotic device has a plurality of movable members to move the device within a body cavity and a control system.

A preferred embodiment of the invention uses a device with at least three members or legs that can be controlled by the user to position the device relative to a region of interest within a body cavity. The device can be configured in a delivery position for insertion into an endoscope channel along with a delivery device to provide for endoscopic insertion.

A preferred embodiment of the invention has a tool interface such that one or more diagnostic or therapeutic devices can be mounted or attached to the interface. Diagnostic components can include imaging devices or sensors to provide images of a region of interest spatial tracking devices to provide localization of the device or sensors to measure characteristics of the tissue. Therapeutic tools can include cutting or suturing devices, tools that can attach to a body surface or that administer a therapeutic agent, monopolar or bipolar electrosurgical device, cryo-cooling elements, laser or other light delivery tools for cutting, cautery, luminal therapy or microwave heating.

A preferred embodiment uses an inflatable bladder system within the members to actuate movement of the device. Each member has a pad, foot or section that can be independently actuated to attach to the surface of the organ or region of interest such as the pericardium. A preferred embodiment utilizes a conforming foot with one or more attachments or suction elements to securely attach the device to the surface.

A preferred embodiment of the present invention involves procedures performed transpericardially, without invasion of the pleural space. Such procedures can include, but are not limited to, cell transplantation, gene therapy for angiogenesis, epicardial electrode placement for resynchronization, epicardial atrial ablation, intrapericardial drug delivery, and ventricle-to-coronary artery bypass, among others.

The ability of the device to move to any desired location in the region of interest from any starting point enables minimally invasive surgery to become independent of the location of the incision. Use of the device also allows a subxiphoid transpericardial approach to any intrapericardial procedure, regardless of the location of the treatment site. As a result, deflation of the left lung is no longer needed, and it becomes feasible to use local or regional rather than general anesthetic techniques. These advantages provide a system for ambulatory outpatient cardiac surgery.

For arrhythmia treatment procedures, the device approaches the heart from the outer surface, placing a walking unit upon the epicardium upon which it moves with the beating heart while navigating across it. The device gains access to the epicardium by crossing through the pericardial sac. The devices uses a minimally invasive approach such as a sub-xiphoid incision combined with endoscopic insertion that provides both visualization during access and a means to safely transect the sac without harming the epicardium. Sub-xiphoid access will place the device initially upon the heart apex to begin its navigation over the cardiac surface. The small size of device, typically 6 mm or smaller in cross section and 20 mm or shorter in length, allows it to use a small diameter access channel to the pericardium, further lessening side effects from tissue damage along the access path to the heart. A preferred embodiment employs a device having dimensions of 10 mm or less in every dimension with a cross sectional diameter of 3 mm or less.

Once the device is within the pericardial sac, it attaches itself to the surface of the heart by means of suction or approaches which provide a connection that keeps the device firmly connected to the epicardium such as, for example, micro-grippers or direct molecular adhesion. Suction holds onto the heart surface and rides with it while having a size small enough to not interfere with normal heart function during the procedure.

The device moves across the surface of the beating heart by having at least two feet that independently make contact with and hold onto the surface. When configured with two feet, the device can move in a manner similar to an inchworm where the front and the back of the device alternately attach to the heart surface and the relative distance between the ends is changed as one of the feet is attached. Thus, with the back foot in place the front can extend away from it while providing the ability to change the direction of movement by pointing the front in desired travel path. When the front finds its attachment, the back foot can detach and contract to bring itself closer to the now attached front foot. When the device is configured with more than 2 feet it can move lateral to the direction it is pointed allowing additional mobility options.

The process by which the device selects its foot and chooses to extend itself is determined based on input from the physician controlling it. They indicate which direction and speed at which the device moves through an intuitive user interface such as a proportional joystick from which the direction and magnitude of the user's pointing action is extracted to control movement. The device finds its own footing by automatically probing in the desired travel direction to achieve effective attachment to the epicardium confirming its new connection to the heart with embedded sensors.

A unique, but common situation, is for the device to encounter fat attached to the epicardium or other internal body surface. In this case, the device's foot configuration allows it to maintain suction upon the fat without tearing it loose from its attachment. The device can detect the presence of fat underfoot by, for example, sensing an impedance change and shift its attachment strategy to achieve this connection without loosening itself or the fat. Another strategy that the device can employ when traversing the heart should the fat prove to be unstable is to maintain an attachment to the pericardial surface while crossing fatty areas. The device can carry this out by having an alternate set of suction connections on the side away from the epicardium which can be used instead of the usual epicardial feet. The device also contains mitigation elements in its suction system to prevent fat from being pulled into its system and plugging it. This includes the specific configuration of the feet and a flushing system that removes the fat should it get into the vacuum system.

A preferred embodiment of the invention uses a rounded and elongated or cylindrical body having a front section and a rear section that move longitudinally with respect to each other. Each section has at least two attachment mechanisms on opposite sides thereof such that each section can attach to the opposite sides of a body cavity or lumen. The attachment mechanisms can be suction elements that are concentrically arranged around the rounded periphery of each section. While the rear section is attached to the walls of the lumen, the front or first section is moved forward. The front section is then adhered to the lumen wall and the rear or second section is moved forward. A central channel can be used to provide control of movement and other operations of the device.

A further embodiment of the invention involves the use of the robot as a remote camera platform to observe a surgical procedure within the abdominal (peritonical) cavity. During certain procedures the abdomen is inflated so that the robot can move across the distended wall and can observe and record the procedure at a distance of up to a few inches. The on-board camera or fiber scope can employ a distally mounted zoom lens so that the depth of focus can be adjusted. The zoom lens can include a fluid lens system. A light source such as an LED array can be mounted on the robot for remote illumination of the field of view.

### BRIEF DESCRIPTION OF THE DRAWINGS

For the present disclosure to be easily understood and readily practiced, the present disclosure will now be described, for purposes of illustration and not limitation, in connection with the following figures:

FIG. 1 is a perspective view of a robotic device in accordance with a preferred embodiment on the invention;

FIGS. 2a and 2b are detailed views of a robotic member with corresponding sectional views in FIGS. 2a-1 and 2b-1;

FIG. 3 is a broken away view of a position tracking system;

FIG. 4 is a view of a robotic imaging sensor;

FIGS. 5a and 5b illustrate another embodiment of a robot movement system according to the present invention;

FIGS. 6a and 6b illustrate a sectioned foot member and a flexible foot respectively;

FIG. 7 illustrates endoscopic delivery of a robotic device in accordance with the invention;

FIG. 8 is a schematic illustration of a control system and interface in accordance with a preferred embodiment of the invention;

FIG. 9 is a schematic perspective view of a cylindrical robot system having first and second sections for movement within body lumens;

FIG. 10 illustrates a side view of a mechanical system for lead placement;

FIG. 11 illustrates a top view of a cable system for external control of rotational movement;

FIG. 12 illustrates a further embodiment providing rotational movement;

FIG. 13 illustrates a control system for suction attachment to a body; and

FIG. 14 illustrates an embodiment of remote control of a robot in accordance with the invention.

### DETAILED DESCRIPTION OF THE INVENTION

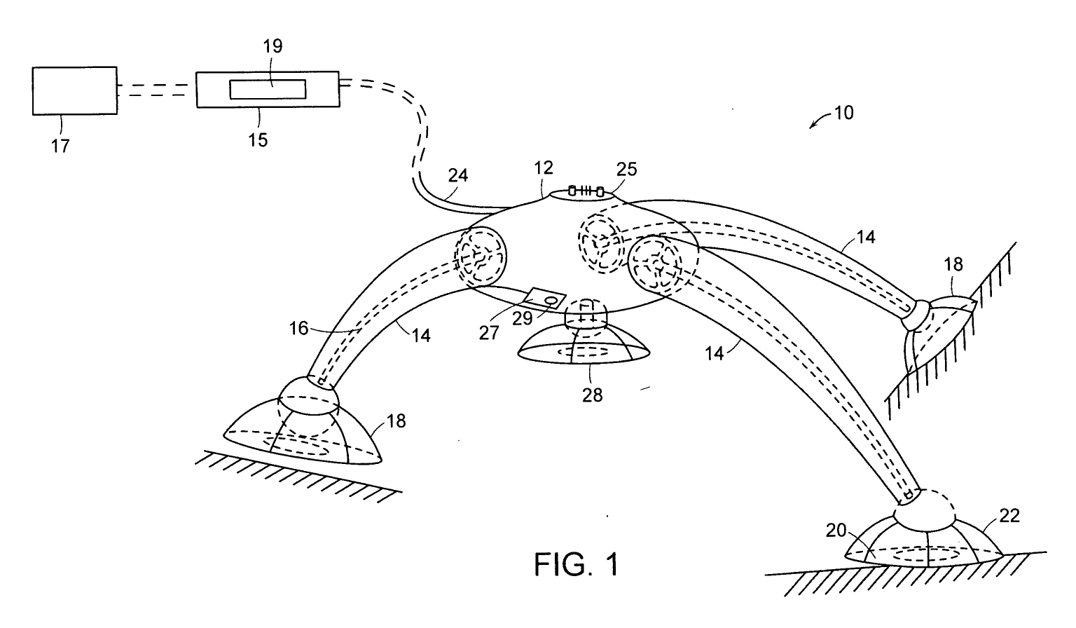

A preferred embodiment of a robot constructed according to the present invention is illustrated in FIG. 1. The device 10 includes forming a central body 12 and a plurality of members or legs 14. The device can have a 6-20 mm cross sectional footprint and a length of 5-20 mm, for example. That size allows the device 10 to fit within a standard 20 mm diameter cannula or endoscope channel. Each of the body sections 14 is equipped with an independent suction line 16 and a foot 18 with one or more suction pad or pads 20, 22, respectively, for gripping to biological tissue. The suction lines 16 and suction pads 20, 22 illustrate a preferred system for prehension.

The translation and rotation of the body section 12 is controlled from an external control system, in this embodiment a handle 15. This can be controlled remotely by RF transmission to the robot and/or by a single or multi-lumen sheath 24. A single or three independently actuated lumens in the sheath 24 provide at least three degrees of freedom for body 12, two angular and one translational. The two angular degrees of freedom allow the device 10 to adapt to the curvature of the heart (or other organ in the gastrointestinal track, for example) as well as turn laterally (i.e. yaw).

Movement is achieved by alternating the actuation level and the suction force exerted by the different legs. With the suction pads in one foot turned on, the suction pads in one or two of the other feet are turned off to allow the device to translate and/or rotate. Forward steps can be taken by repeating the process. Turning can be achieved by differentially actuating the legs. The actuation of the lumens at the handle may be performed manually, along with the opening and closing of the valves to the suction lines. Actuation of the device can also be performed under computer control.

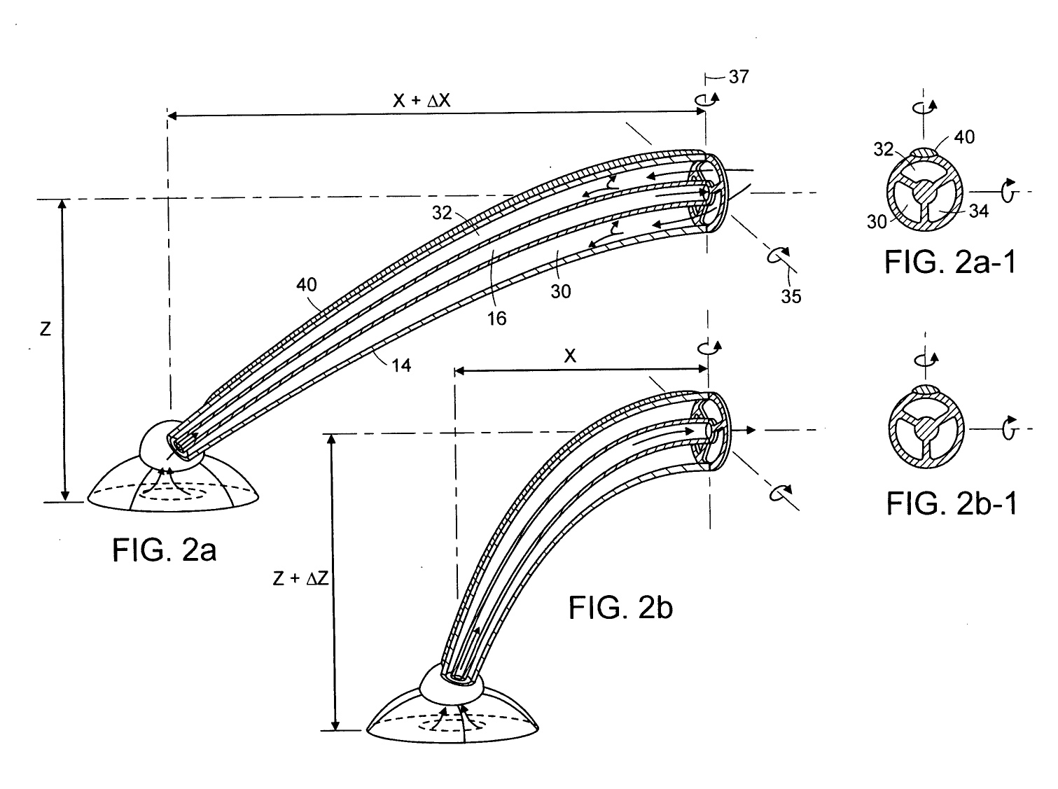

Sectional views of a preferred embodiment of the invention are illustrated in FIGS. 2a and 2b. Member 14 has channels 30, 32, 34 that can be pressurized by connection to a pressurized gas or fluid source 17. Sheath 24 can have one or more lumens to couple the channels 30, 32, 34 to the source 17. When the member 14 is pressurized it extends the length by an amount ΔX. By differing the pressurization of channels 30 and 34 the member can rotate around axis 37. The pressure can also be varied to cause rotation about axis 35 to cause a change in elevation ΔZ. Varying pressurization simultaneously in multiple lumens can change the stiffness of the device with any combination of feet attached to the tissue. Sectional views in FIGS. 2a-1 and 2b-1 show the lumens or channels 30, 32, 34 and spring member 40 which provides a resting position for member 14. Systems and methods for manual control are described in greater detail in U.S. application Ser. No. 10/982,670 filed on Nov. 5, 2004, the entire contents of this application being incorporated herein by reference.

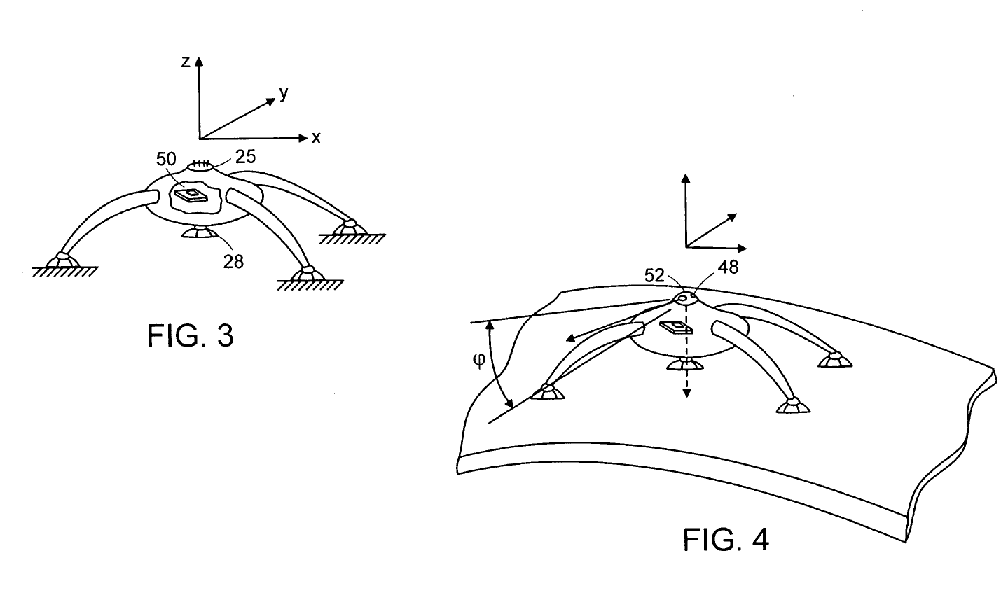

Shown in FIG. 3 is a location sensing tracking device 50 or marker so that the position or orientation can be identified electromagnetically or under fluoroscopy. A tool or sensor device can be mounted on one or more tool interface fixtures 25. Such a fixture can also be mounted underneath the device adjacent to the suction element 28 that stabilizes the tissue adjacent to or around the tool or sensor. One or more corking channels 27 can allow the user to insert tools or fiberscopes 29 on the underside of the device. One or more suction or gripping devices can also be placed on the top of central body 12, that is, on the opposite side from element 28, to provide for attachment of the second side of the device to a second cavity wall or surface. The sensor can be mounted directly under each suction element, for example, to measure the contact pressure or to detect the presence of an artery or other organ feature that should not be a location for attachment. The sensor can measure temperature, pH, detect impedance to discriminate between tissue types or provide for optical sensing.

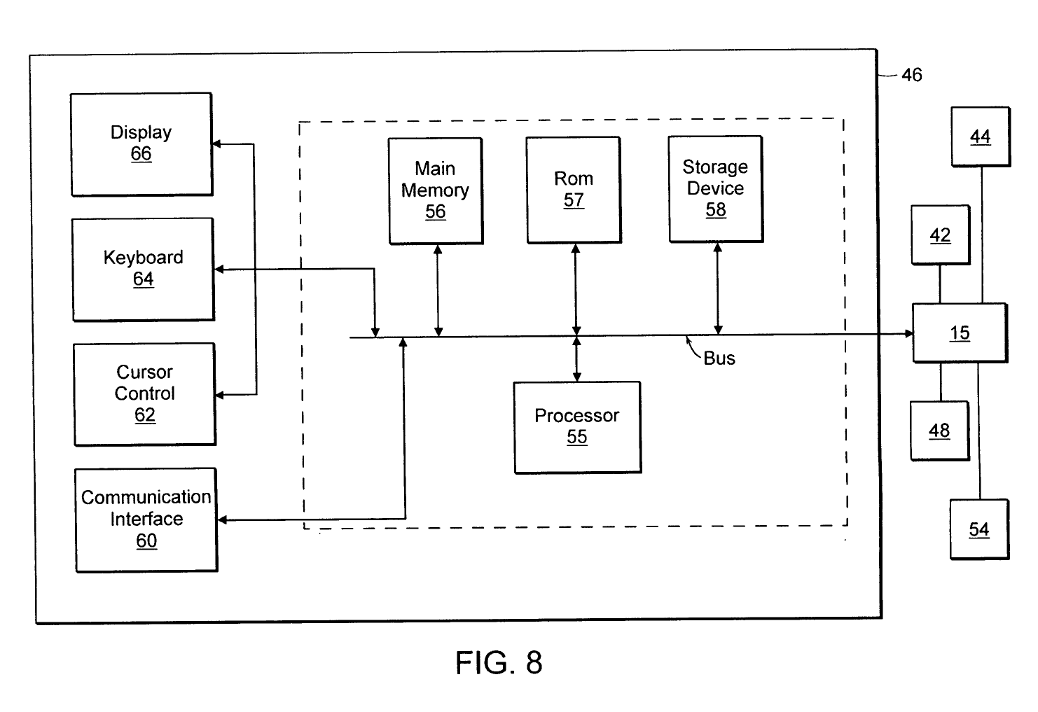

Additionally, a fiberscope 52 (FIG. 4), running through the length of the sheath 24, may be fixed on the body 12 to provide visual feedback, with or without the use of an adjustable mirror 40. The images from the fiberscope 52 may be captured with a digital video camera 42 (See FIG. 8) and displayed as a part of the graphical user interface (GUI) on display 66, both of which are part of a control system 46. The control system 46 may include sensors 48 for monitoring the vacuum supplied by suction lines 16, electronically controlled valves for determining which suction pad 20, 22 is operative, and a vacuum source 44. An image sensor can be mounted at 52 and can be connected by wire or wirelessly to the control system. The control system 46 may also include motors 54 for controlling actuation of the robot. A controller or computer processor 55 may be provided to control the various components in response to information input by the user via the GUI, keyboard 64, cursor 62, interface for a network 60 or handle control pad 19, electromagnetic sensor or haptics feedback to sense slipping and to control locomotion and other functions. This allows for the motors 54, solenoid valves, etc. to be located outside the device 10. Thus, in a preferred embodiment, there are no electrically active components in device 10, the device having only mechanically actuated components. The robot 10 may be either a disposable device or a reusable, sterilizable device.

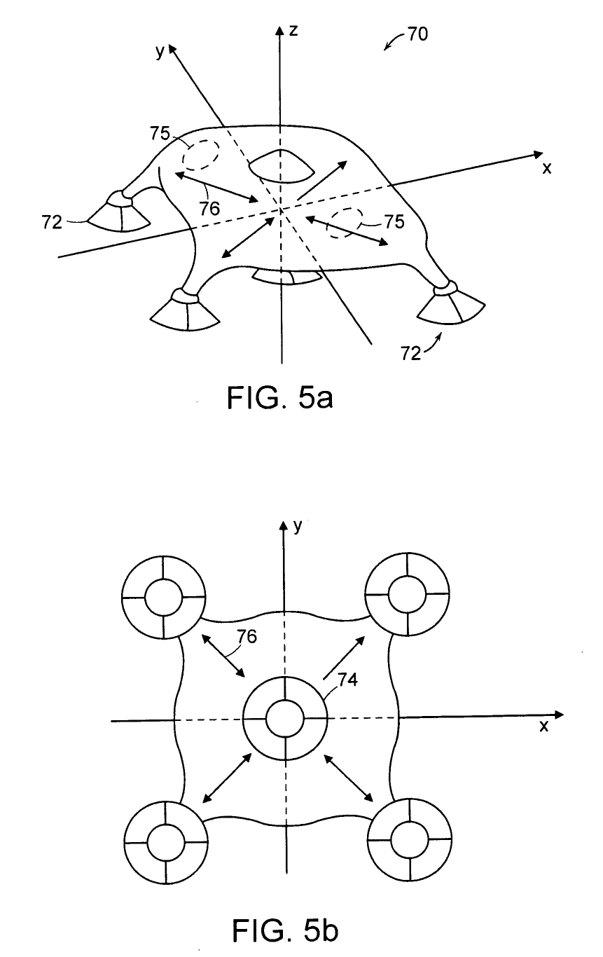

FIGS. 5a and 5b illustrate a preferred embodiment of the invention in which internal bladders or actuators are used to propel the device 70. Each of four sections can be actuated to move a respective section along a given axis 76 to actuate movement. Suction elements can be housed within body 70.

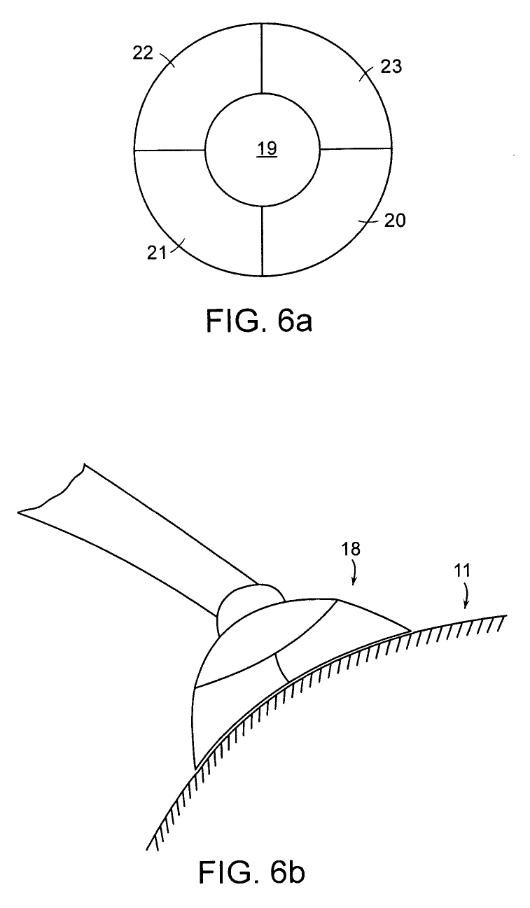

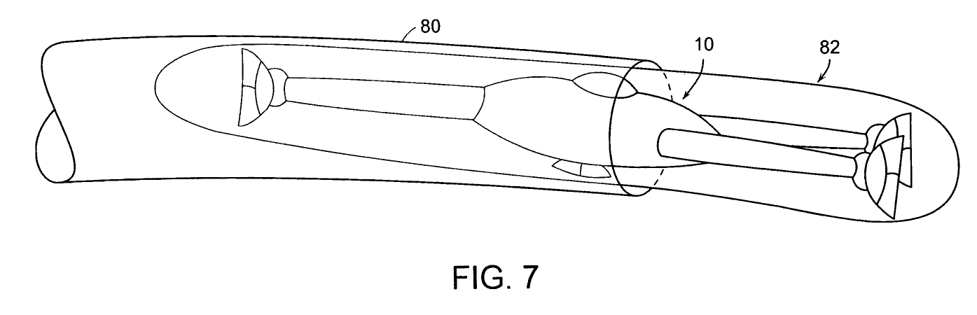

In the embodiment of FIG. 6a, the suction pads 19-23 are connected to the bodies 12, 14 using feet 18 of varying flexability, respectively. That enables the suction pads 19-23 more freedom to conform to a curved surface 11 of the organ as shown in FIG. 6b. Meshes may cover the bottom of the suction pads to keep out large particles, while suction filters or other devices can be provided to remove fluids and small particles.

An aspect of the present invention is changing the frame of reference of the robot from that of the user or physician to that of the moving organ. For example, although in the disclosed embodiments movement is achieved through the actuation of member 14, either manually or through the activation of motors, other methods such as local (i.e. positioned on the robot) electric motors (operated with or without a tether), or local ultrasonic motors (operated with or without a tether) can be used. The means for prehension in the disclosed embodiment is suction. Alternative means of prehension may include microgrippers, molecular adhesion, synthetic gecko foot hair or a “tacky” foot. The actuation for treatment may include all the same alternatives as for robot movement. Finally, the device may operate with a tether having wires and pneumatic or fluidic lines as disclosed above, with a tether having electric wires for local motors or video from a camera, or the device may operate without a tether. Tethered devices can have mechanical control wires that can be manually rotated, inserted or withdrawn to either control movement of the robot or operate a tool. Tetherless models can be powered by a battery, the transcutaneous charging of a coil, etc., and can be controlled by local computing or through radio frequency or magnetic transmissions. It will be understood by those of ordinary skill in the art that changing the frame of reference of the robot from that of the user to that of the moving organ can be brought about by a wide variety of robots designed so as to be able to move within a body cavity. A body cavity refers to that space surrounding an organ such as, for example, the peritoneal space surrounding the liver, the pleural space surrounding the lungs, the pericardial space surrounding the heart, etc.

A tool such as a needle can be carried within a recess in body 12. Body 12 can also carry tools for providing images such as a fiberscope or camera, with or without some combination of lenses or mirrors 40, fiberoptics, etc. The needle may used to perform epicardial electrode lead placement for cardiac resynchronization therapy (CRT) via subxiphoid videopericardioscopic access. A robot 10 equipped with the needle can perform a minimally invasive suturing technique that can be used with a variety of epicardial pacing leads, both permanent and temporary. A minimally invasive forceps, passing through an off-center working port of the robot 10 can be used to grasp objects.

The robot 10 can have a separate electrode channel that allows passage of the electrode and its wire lead from outside the body into the pericardium to be attached to the heart by screw in leads or barbed leads. The needle, forceps, wire “fork”, suture with sharpened cap, and all supporting instrumentation needed for a suturing technique to attach the leads can be sterilizable or disposable. Actuation of a tool may be performed locally by motors inside the robot, or from outside the body using a wire running through the cannula. Visual feedback for a procedure can be provided by the same device used during positioning.

Turning to FIG. 7, in operation according to one aspect of the present invention, the device 10 will enter the pericardium and be placed on the epicardial surface of the heart using a rigid or flexible endoscope with a working port. The endoscope can be introduced into the pericardial sac through a port or limited incision beneath the xiphoid process of the sternum.

Once positioned appropriately with the endoscope under direct visual confirmation, the device 10 grasps the epicardium using suction. The suction forces are applied through the independent suction pads 19-23 that may be attached directly to member 14 or through compliant or flexible feet 18. The vacuum pressure is supplied to the suction pads 19-22 by the vacuum source through the operation of valves and suction lines 18 respectively. The vacuum source provides a variable vacuum pressure with 0.08 N/mm², being effective and safe for use in FDA approved cardiac stabilizers. The suction forces generated by this pressure have proven effective for our application, and did not damage the epicardial tissue. During movement, the vacuum pressure is monitored by the external pressure sensors and regulated by computer-controlled solenoid valves, both located within the control system 46.

The device 10 provides visual feedback to the user during movement and administration of therapy. That can be accomplished using fiberoptics to relay the image from the device 10 to the camera 42 located in the control system 46. Alternatively, a CCD video camera can be mounted directly to the device 10. This provides all of the necessary vision with a single visual sensor on a fixed mount. Alternatively, either the viewing head can be actuated for motion, or two imaging devices can be incorporated: one tangential to the surface of the organ (looking forward) for providing information for navigation, and the other normal to the surface (looking down) for providing a view of the area to receive attention, e.g. treatment, testing, etc.

Diagnostic methods or therapies administered from the device 10 do not require stabilization of the heart because the device 10 can be located in the same reference frame as the surface of the heart, rather than that of a fixed operating table. This eliminates the need for either endoscopic stabilizers, which require additional incisions, or cardiopulmonary bypass, which increases the complexity and risk of the procedure.

The teleoperative surgical systems in use today utilize laparoscopic manipulators and cameras and are introduced to the pericardial sac through several intercostal (between rib) incisions. These instruments must then pass through the pleural space before reaching the heart, which requires the collapsing of a lung. The delivery of the device 10 onto the heart does not require collapsing a lung because it can be introduced to the thoracic cavity through an incision made directly below the xiphoid process. The endoscope will then be pushed through the tissue and fascia beneath the sternum until the surface area of the pericardium is reached, never entering the pleural space. The scope can also be used to breach the pericardium, thus delivering the device 10 directly to the epicardium. Because the device 10 does not require the collapsing of a lung, it does not require differential ventilation of the patient, and it is therefore possible that local or regional anesthesia can be used instead of general endotracheal anesthesia (GETA). As a result, a potential benefit is that the device 10 may enable certain cardiovascular interventions to be performed on an ambulatory outpatient basis.

The capabilities of the device 10 enable it to reach virtually any position and orientation on the epicardium. This is not the case with rigid laparoscopes, which are limited to a relatively small workspace near the entry incision. In addition, these systems require the removal and re-insertion of the tools to change the operative field within a single procedure. The device 10, on the other hand, can easily change its workspace by simply moving to another region of the heart.

Beyond issues of achieving effective connection and movement across the heart surface, the device is able to reach all the areas where it needs to treat tissue to produce an effective result. The space between the heart outer surface and the surrounding anatomy, while typically satisfactory to move about on the anterior and left sides, can be limited on some aspects. To provide additional space to allow the device sufficient access to the epicardium, at least two approaches are available. The patient's orientation on the operating table relative to gravity can be adjusted to allow the heart and surrounding anatomy to shift and provide additional space. In addition, a partial bypass can provide additional space around the heart since a side effect of this is that the heart size decreases as its flow output decreases.

With these movement procedures the device is able to achieve reliable motion across the epicardium to carry out ablation of heart tissue, for example. Achieving transmural lesions of the myocardium is important for blocking charge propagation and redirecting current flow to mitigate arrhythmias. This has proven to be a difficult task for epicardial energy delivery systems especially when used in a minimally invasive procedure. However, by decreasing cardiac flow rate through a partial bypass, it is possible to decrease the thermal energy transfer loss and increase the amount of energy which remains in the tissue to produce lesions. This flow moderation can be carried, out using minimally invasive bypass devices.

When the device reaches the specific site where it needs to create a transmural lesion, for example as part of an ablation procedure to treat arrhythmia, it must have available to it appropriate energy delivery tools to do so. Typical energy deliver systems are designed to limit the number of separate lesions must be created because of the difficulty in accurately placing and holding these devices upon the beating heart. Thus, current systems tend to have elongated configurations that can be articulated to deliver energy over large lengths. The present invention due to its stable placement on the heart and its capacity to move while creating lesions, is better suited to energy delivery that is more narrowly focused. Ablation procedures involving multiple small lesions can be performed. Thus compact energy delivery systems such as optical fiber-transported laser energy combined with, for example, deflectable mirrors mounted upon the device.

With sufficient access to the areas that require lesions and availability of tools and techniques to make them, the knowledge of where to precisely place the lesions relative to the charge propagation anomalies needs to be integrated with device navigation. This can be carried out through a number of approaches, e.g. electromagnetic tracking combined with 3D medical imagery, which locate the device's position and orientation relative to known anatomic details or fiducials. These approaches can also provide effective knowledge of the device's location without the need for traditional ionizing radiation based imaging which provides a significant advantage for physicians and patients over endovascular approaches that can use more than 4 hours of fluoroscopy time for a single procedure.

With selected tools, the device is able to perform epicardial cardiac procedures such as: cell transplantation, gene therapy, atrial ablation, and electrode placement for resynchronization and myocardial revascularization. Devices such as an ultrasound transducer, diagnostic aid or other sensor, drug delivery system, therapeutic device, optical fiber, camera or surgical tool(s) may be carried by the device 10. Additionally, procedures on living bodies other than humans, e.g. pets, farm animals, race horses, etc. can be used while remaining within the teachings of the present invention.

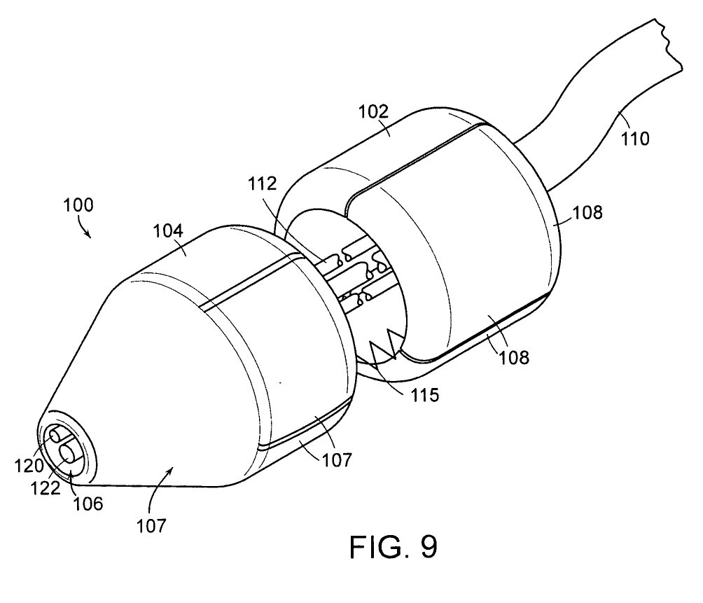

FIG. 9 illustrates another preferred embodiment of the invention that can be used in tubular lumens within the body such as the bronchi of the lungs, the gastrointestinal tract including the colon, the spinal column, and ventricles of the brain. In this embodiment, a device 100 can comprise first 104 and second 102 sections that both have a rounded generally cylindrical shape. Each section has two or more peripherally arranged attachment devices 107, 108, such as, suction elements, each of which can be separately controlled. Thus while the rear section 102 is attached to the walls of a body channel the first section 104 can be advanced along the channel. The sections can be moved by longitudinally rigid cables 112, one or more pressurized bellows or bladders 115 that connect the two sections (see e.g. www.shadowrobot.com/airmuscles). The bellows or bladders can have three or more wire guides that can extent through the sheath to external controller and thereby control the direction in which the device is moved.

The robot 100 can have a cone 107 on the first section 104 to provide a shape that gradually widens a constricted channel. The cone can have a distal aperture or opening 106 through which tools can be passed or to provide viewing with a camera or a fiberscope 122. A wire 120 for lead placement or other tool or sensor can be positioned in or moved through the opening 106. A single sheath 110 can be used to connect all the control elements to an external controller. The sheath can be a reduced diameter relative to device 100.

The sections 102, 104 can also have internal pressurized bladders that can expand or contrast to control the diameter of the device to bring suction elements 107, 108 into contrast.

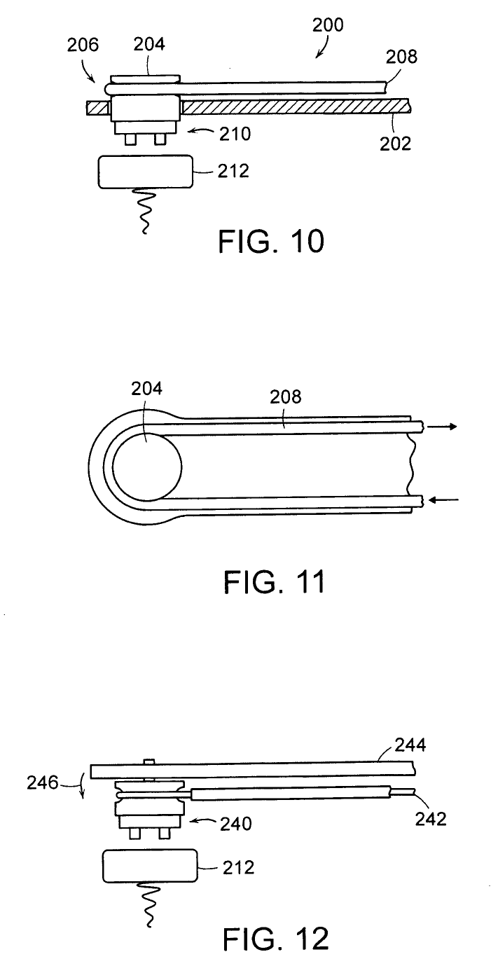

FIG. 10 shows a tool assembly 200 that can be mounted to the various embodiments requiring rotation. In this embodiment, a structural member 202 holds a rotating shaft 204 with a notched spool 206 above the member that is rotated using a cable 208. By pulling on the cable, a connector 210 that rotates with the spool can be connected to a screw-in lead 212, for example.

FIG. 11 shows a top view of the cable 208 and pulley system. FIG. 12 illustrates another embodiment in which the connector 240 is directly driven by the cable 242. A nitinol support structure can be used to hold the connector. The nitinol can be pretensioned to assume a particular angle 246 relative to the tissue so that the lead enters at a desired angle.

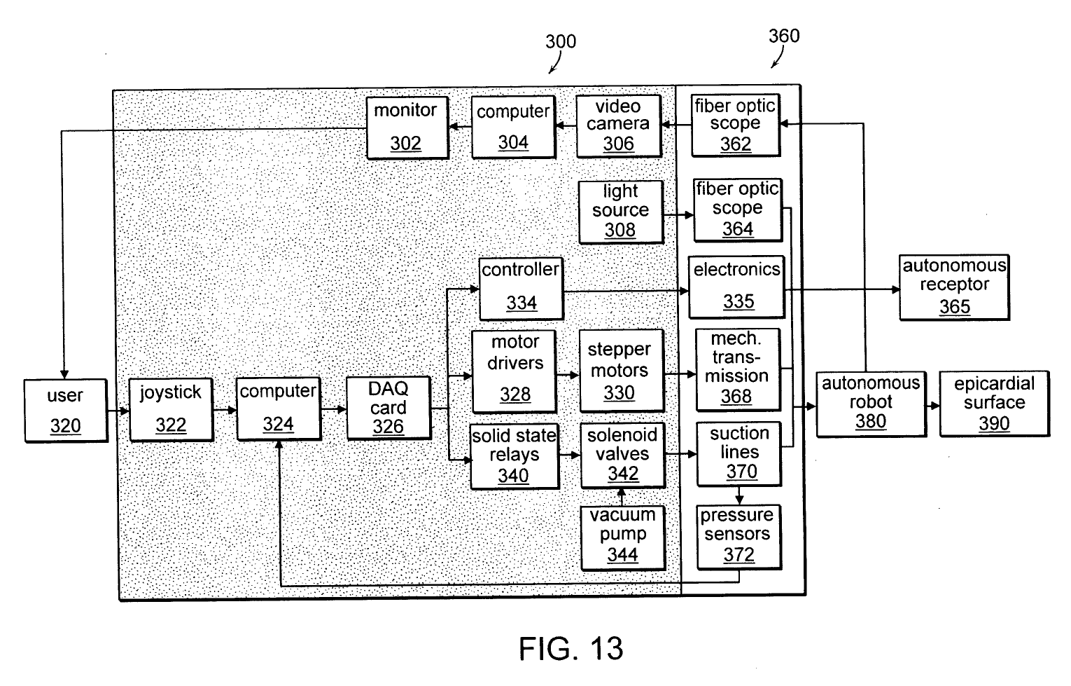

FIG. 13 illustrates a schematic diagram of a control system for a tethered robot in accordance with the invention. The tether or cable 360 includes a fiberoptic scope having a plurality of fibers 364 connected to a light source 308 which can be a white light source, or other broadband light source, or a laser for therapeutic or diagnostic applications. A second plurality of fibers 362 can be connected to a video camera 306. The user control system 300 allows the user 320 to manipulate the joystick 322 to instruct computer 324 which sends control signals through card 326 to the control 334 for wired or wireless connection 335 to receiver in either a tethered device 380 or an autonomous device 365. The control signals also can be used for robotic control of mechanical systems with drivers 328, motors 330 and coupling 368. Control signals also control relays 340, valves 342 with a pump 344 to manage suction lines 370 which are monitored with sensors 372. The user can also manually control mechanical cables directly through the sheath 360 to provide movement on the tissue surface 390.

The camera 306 can be connected to computer 304 and monitor 302 for viewing.

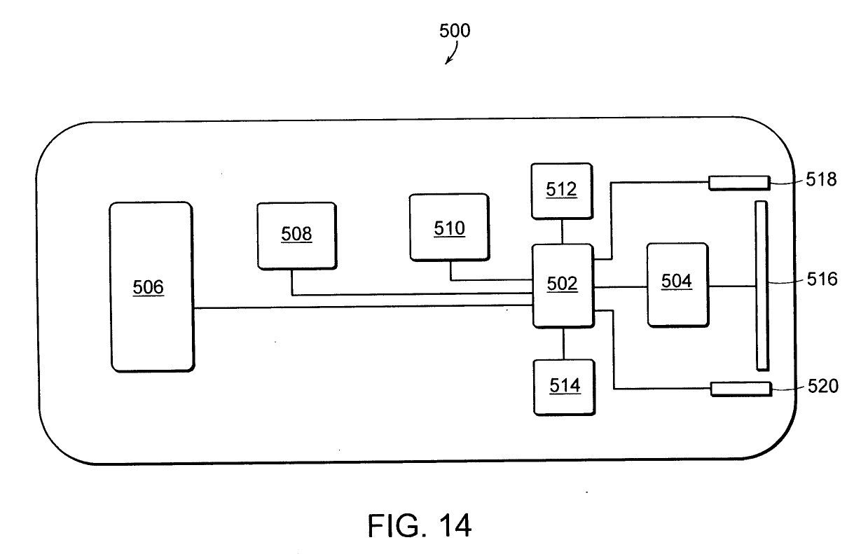

FIG. 14 shows an on-board schematic diagram of a control system for a tetherless robot. In the tetherless system 500 can include a camera 516 connected to readout circuit 504 that is connected to the device controller or processor 502. A battery 506 provides power to the system which uses an antenna 512 to transmit image data and receive instruction. The controller also actuates light source elements 518, 520 that illuminate the field of view which can emit light in the visible or infrared ranges for multispectral viewing. The controller also actuates movement through activation 514 and receives data from sensor elements 508 and actuates drug delivery 510 for external needle or needle arrays.

Thus, while the present invention has been described in connection with preferred embodiments thereof, those of ordinary skill in the art will recognize that many modifications and variations are possible. The present invention is intended to be limited only by the following claims and not by the foregoing description which is intended to set forth the presently preferred embodiment.

What is claimed is:

1. A robot for insertion into a body cavity, comprising:
a robotic device having a central body and a plurality of movable members to move the device within the body cavity; and
a control system for controlling the robotic device.

2. The robot of claim 1 further comprising an interface that the control system with the device.

3. The robot of claim 1 further comprising a prehension device that includes at least one of a suction pad, synthetic gecko foot hair, or a tacky foot.

4. The robot of claim 2 wherein said interface includes a one of a plurality of wires, cables or flexible drive shafts.

5. The robot of claim 4 wherein said control system includes a handle and said wires, cables or flexible drive shafts are carried, for at least part of their length, in a sheath.

6. The robot of claim 1 wherein said robot is sized to fit within a 20 mm diameter channel in a delivery configuration.

7. The robot of claim 1 further comprising a tool including one of an ultrasound transducer, a diagnostic sensor, drug delivery system, therapeutic device, or surgical tool.

8. The robot of claim 1 further comprising at least one of a camera or fiberscope.

9. The robot of claim 8 further comprising a position sensing device to provide navigation information.

10. The robot of claim 8 further comprising a second camera or fiberscope.

11. The robot of claim 1 further comprising a plurality of suction elements.

12. The robot of claim 1 further comprising a lead wire that connects to heart tissue.

13. The robot of claim 1 wherein the plurality of movable members comprises a plurality of independently controlled legs.

14. The robot of claim 12 wherein each leg has a pad that contacts a tissue surface.

15. The robot of claim 13 wherein each leg comprises an inflatable bladder.

16. The robot of claim 13 wherein each leg has a plurality of inflatable bladders, each bladder being in fluid communication with a fluid pressure control system.

17. The robot of claim 14 wherein each pad has a suction element.

18. The robot of claim 17 wherein each pad has a plurality of at least three controllable suction elements.

19. The robot of claim 1 further comprising a channel for delivery of a therapeutic agent.

20. The robot of claim 1 further comprising a tool interface for mounting a tool on the robot.

21. The robot of claim 1 further comprising a lead placement tool mounted on the robot.

22. The robot of claim 1 further comprising a rotating tool element mounted on the robot.

23. The robot of claim 1 further comprising a cutting tool.

24. The robot of claim 1 further comprising a tissue ablation tool.

25. The robot of claim 14 wherein the tissue ablation tool comprises a laser.

26. The robot of claim 1 further comprising a laser light source.

27. The robot of claim 26 wherein the laser light source is coupled to the robot with a fiber optic cable.

28. The robot of claim 1 further comprising a broadband light source.

29. The robot of claim 28 wherein the broadband light source is coupled to the robot with a fiber optic cable.

30. The robot of claim 1 further comprising a needle or needle array.

31. The robot of claim 1 wherein the robot is less than 10 mm in diameter.

32. The robot of claim 1 further comprising a cable sheath connecting the robot to a controller.

33. The robot of claim 1 further comprising a body having a first section with a first plurality of attachment members and second section with a second plurality of attachment members.

34. The robot of claim 33 wherein the attachment members are suction elements.

35. The robot of claim 34 wherein the suction elements are connected by valves to a vacuum source.

36. The robot of claim 33 wherein the sections are cylindrical.

37. The robot of claim 33 wherein the attachment members are on opposite sides of the robot.

38. The robot of claim 1 wherein the robot comprises a walker having a plurality of legs.

39. The robot of claim 1 wherein the robot has a size for insertion in bronchi of the lung.

40. The robot of claim 1 wherein robot has a size for insertion into a spinal column.

41. The robot of claim 1 wherein the robot has size for insertion into a grain ventricle.

42. The robot of claim 1 wherein the robot has a working channel for manual insertion of a tool.

43. The robot of claim 33 wherein the first and second sections are connected by a pressurized bellows.

44. The robot of claim 1 wherein the device has a distal opening for viewing of a field of view.

45. The robot of claim 1 further comprising a cable that rotates a tool.

46. The robot of claim 1 further comprising a wireless connection between the robot and a controller.

47. The robot of claim 1 further comprising an electronic sensor for measuring a tissue characteristic.

48. The robot of claim 1 further comprising a battery in the robot.

49. The robot of claim 1 further comprising an electronic control system in the robot.

50. A robot for use in a living body, the robot comprising a central body and a plurality of movable members, each movable member having a prehension device that can attach the robot to an organ.

51. The robot of claim 50 wherein said robot members include a suction element.

52. The robot of claim 50 wherein said robot includes two body sections, each body section carrying one of suction pads, synthetic gecko foot hair, or a tacky foot.

53. A method of positioning a robot on an organ within a living body, comprising:
placing the robot on an organ, the robot having a central body and a plurality of movable members;
affixing the robot to the organ so the robot is in the same frame of reference as the organ; and
moving the robot along the organ while remaining in the same frame of reference as the organ.

54. The method of claim 53 further comprising providing a pressurized gas source that is connected to the robot with a cable.

55. The method of claim 53 further comprising providing a conformable suction pad having a plurality of suction elements.

56. The method of claim 53 further comprising providing a member having an inflatable channel.

57. The method of claim 53 further comprising providing a fixture on the device for mounting a tool or sensor.

58. The method of claim 53 further comprising inserting the robot onto a surface of the heart within the pericardial sack and attaching a lead to the heart with a screw or barbed lead.

59. The method of claim 53 further comprising distending a bodily cavity and viewing a medical procedure in the cavity with a camera coupled the robot.

60. The method of claim 53 further comprising adjusting a field of view of a camera coupled to the robot with a zoom lens.
